(19) United States
(12) Patent Application Publication
(10) Pub. No.: US 2026/0295228 A1
(43) Pub. Date: Oct. 1, 2026

(54) SYSTEM FOR SYNCHRONIZED INSTRUMENT AND FLUID DEPLOYMENT

(71) Applicant: IotaMotion, Inc., St. Paul, MN (US)

(72) Inventor: Constantinos Nikou, Monroeville, PA (US)

(21) Appl. No.: 19/635,318

(22) Filed: Mar. 31, 2026

Related U.S. Application Data
(60) Provisional application No. 63/781,715, filed on Apr. 1, 2025.

(51) Int. Cl. A61M 31/00; A61B 10/00

(52) U.S. Cl. CPC A61M 31/00; A61B 10/0045; A61B 2010/0054; A61M 2202/0007; A61M 2202/0014; A61M 2202/04; A61M 2205/3327; A61M 2205/3379; A61M 2205/502; A61M 2210/0662

(57) ABSTRACT

Synchronized instrument and fluid deployment is achieved through coordination of translation of a medical instrument with distribution of a fluid or withdrawal of biological matter. A schedule is executed to deploy the medical instrument a first distance within a patient's anatomy, distribute a first portion of the fluid, translate the medical instrument a second distance, and distribute a second portion of the fluid. The schedule is stored in memory, and instructions are executed by processing circuitry to transmit control signals to an insertion actuator and an instrument actuator. This enables precise control over the deployment of the medical instrument and the distribution of the fluid or withdrawal of biological matter, facilitating various medical procedures, such as those involving the inner ear.

### CLAIM OF PRIORITY

This patent application claims the benefit of priority, under 35 U.S.C. Section 119(e), to Constantinos Nikou U.S. Patent Application Serial Number 63/781,715, entitled “SYSTEM FOR SYNCHRONIZED INSTRUMENT AND FLUID DEPLOYMENT,” filed on Apr. 1, 2025, (Attorney Docket No. 4700.012PRV), which is hereby incorporated by reference herein in its entirety.

### TECHNICAL FIELD

Examples described herein generally relate to medical instruments and, more specifically, to a system for synchronized instrument and fluid deployment.

### BACKGROUND

The inner ear, also known as the labyrinth, is located within the temporal bone of the skull and contains both bony and membranous labyrinths. The inner ear can include three distinct regions: the cochlea for hearing, the vestibule for balance, and the semicircular canals for detecting rotational movements. Clinicians perform various medical procedures within the inner ear to treat various issues with hearing, balance, and other related ailments.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates a schematic diagram of a system for synchronized instrument and fluid deployment.

FIG. 2 illustrates a schematic block diagram showing an example of synchronized instrument and fluid deployment.

FIG. 3 illustrates a block diagram of an example of a user interface and a data structure for synchronized instrument and fluid deployment.

FIG. 4 illustrates a flowchart of an example of a method for synchronized instrument and fluid deployment.

FIG. 5 illustrates a block diagram of an example of a machine upon which one or more examples can be implemented.

### DETAILED DESCRIPTION

Delivering therapeutic fluids to the inner ear, such as viral vectors for cochlear gene therapy, or sampling biological matter such as perilymph fluid, can involve manual coordination of instrument insertion and fluid transfer by a clinician. Manual coordination can result in inconsistent insertion depths, uneven fluid distribution across the cochlea, uncontrolled fluid transfer rates, and physical contact between the instrument and cochlear structures. Uncontrolled fluid transfer rates can induce turbulence in the perilymph fluid, which can displace the delivered therapeutic agent from the target location. Uneven distribution can result in portions of the cochlea receiving insufficient therapeutic agent while other portions receive excess. These issues can be compounded by the small scale of the cochlear anatomy and the limited access available to the clinician. A system that synchronizes the translation of a medical instrument with the distribution of fluid or withdrawal of biological matter according to a defined schedule can address these issues. The schedule can coordinate the position of the medical instrument with the volume and rate of fluid transfer at each position, enabling distribution of fluid at multiple locations along the cochlea. The system can use a tracking device to monitor the insertion depth of the medical instrument and provide position feedback to a processor that controls both an insertion actuator and an instrument actuator. By synchronizing instrument translation and fluid transfer according to the schedule, the system can control the rate of fluid delivery to limit turbulence in the perilymph fluid and can distribute the fluid across defined locations within the cochlea rather than at a single point.

Fluid transfer capabilities can be utilized for applications in the inner ear, such as gene therapy delivery or perilymph fluid sampling. Cochlear gene therapy can involve injecting a viral vector into the cochlea at multiple locations. The needle can be a flexible needle. The needle can be attached to a flexible lumen. The flexible needle or the flexible lumen can be inserted into the cochlea to a set depth. When the flexible needle or flexible lumen is inserted into the cochlea to the set depth, a portion of the fluid can be delivered to the cochlea via the flexible needle or lumen using a syringe. The amount of fluid delivered to the cochlea can be controlled. A mechanized pump or syringe can be used to control the amount of fluid delivered to the cochlea.

The actuation of the needle and the delivery of the fluid can be configured to limit physical contact with the cochlea. After a first amount of the fluid is delivered to the cochlea, the needle can be withdrawn a defined distance, and the dose of fluid can be repeated. The dose of fluid can be distributed during a controlled withdrawal of the needle. The withdrawal of the needle and the distribution of the fluid can be controlled to ensure the fluid is evenly distributed throughout the cochlea. The distribution of the fluid can be controlled at a rate to limit turbulence in the perilymph fluid of the cochlea. Limiting turbulence in the perilymph fluid of the cochlea can facilitate uniform distribution of fluid within the cochlea.

A system can be configured to sequence or synchronize the injection and movement of the needle. In some applications, perilymph fluid within the cochlea can be sampled to detect localized disease or monitor therapeutic processes. A system can be configured for sequencing or synchronizing fluid withdrawal and needle movement. Whether inserting a fluid or withdrawing biological matter, the device can be configured to limit turbulence within the cochlea, thereby controlling the distribution of fluids and reducing physical contact with the cochlea.

The system can include a control system configured to synchronize the actuation of an instrument, such as a needle, a flexible needle, a flexible lumen, or a catheter, and the distribution of fluids, such as a viral vector, or the withdrawal of biological matter, such as perilymph fluid, tissue, or infection. The system can integrate motorized lumen control with controlled injection machines by coupling them through a controller interface. The controller interface can accept runtime or preprogrammed motion and injection pairings from the user for automated fluid delivery or sampling.

A system for controlled delivery of biotherapeutics to the inner ear can include a medical instrument that can be inserted and retracted by a lumen insertion actuator. The lumen insertion actuator can be configured to receive commands from a controller or a processor and can be removably coupled to the insertion mechanism.

The system can include an injector actuator. The injector actuator can regulate the distribution or administration of fluids, such as stem cells or gene therapies, or the withdrawal of biological matter, such as perilymph, infection, or tissue. The system can include tracking devices, such as encoders, step counters, linear variable differential transformers, or combinations thereof, to monitor the insertion depth of the medical instrument relative to the cochlea.

A controller or processor can transmit control signals or commands to the actuator. The control signals or commands can be guided by an input module. The input module can operate based on an injection schedule that specifies injection amounts over specific time periods, insertion or retraction distances over defined timeframes, simultaneous insertion or retraction and injection actions, or alternating patterns of insertion or retraction and injection. The control signals or commands can be guided by an output module. The output module can operate according to a withdrawal schedule that specifies withdrawal amounts over specific time periods, insertion or retraction distances over defined timeframes, simultaneous insertion, retraction, and withdrawal actions, or alternating patterns of insertion, retraction, and withdrawal.

The system can include a user interface. The user interface can enable a user of the system to define the injection schedules or withdrawal schedules. The user interface can enable a user of the system to load the injection schedules or withdrawal schedules from predetermined data files. The user interface can enable users to generate injection or withdrawal schedules based on patient-specific parameters. The patient-specific parameters can include anatomical measurements, patient imaging, disease information, diagnostic data, genetic factors, or combinations thereof.

The system can incorporate an actuator positioner that can be fixed to at least one of the patient or the lumen insertion actuator. The positioner can provide at least two degrees of freedom for adjusting the lumen insertion actuator relative to the target area, such as the cochlea or other anatomy of the patient.

The system can enable clinicians to deliver therapeutic agents to the inner ear according to defined parameters. The system can enable clinicians to withdraw biological matter from the inner ear. The control provided by the system can facilitate treatment for auditory disorders.

Although examples described herein reference the cochlea and inner ear anatomy, the system and methods described are not limited to inner ear procedures. The synchronized control of instrument translation and fluid transfer according to a defined schedule can be applied to other anatomical regions and medical procedures where controlled distribution of a fluid or withdrawal of biological matter along a path within a patient is performed. For example, the system can be used for intratumoral injection of therapeutic agents, where a needle is inserted into a tumor and a fluid such as an immunotherapy agent, a chemotherapeutic agent, or an oncolytic virus is distributed at multiple locations along the needle path during controlled retraction to distribute the agent throughout the tumor volume. The system can be used for spinal or epidural procedures where a fluid is delivered at controlled intervals along a defined length of the spinal canal. The system can be used for intra-articular injection into joints, where a therapeutic fluid is distributed at multiple depths within a joint space. The system can be used for biopsy procedures where biological matter is sampled at multiple depths within tissue along a defined path, and the sampling is synchronized with translation of the instrument to collect location-specific samples. The system can be used for ophthalmic procedures involving delivery of gene therapy vectors or other therapeutic agents to the retina or vitreous cavity, where the anatomy imposes similar constraints on instrument movement and fluid transfer rates. The system can be used for neurosurgical procedures where a catheter or needle is advanced into brain tissue and a therapeutic agent is distributed at defined intervals along the insertion path. In each of these applications, the schedule can be configured to specify fluid volumes, time intervals, translation distances, cycle counts, and mode indicators (simultaneous or alternating translation and fluid transfer) appropriate to the target anatomy and therapeutic objective. The tracking device can monitor instrument position relative to the target anatomy, and the processor can synchronize the insertion actuator and instrument actuator according to the schedule to control the spatial distribution of the fluid or the locations from which biological matter is withdrawn.

The above discussion provides an overview of the subject matter of the present patent application. It is not intended to provide an exclusive or exhaustive explanation of the invention. The description below is included to provide further information about the present patent application.

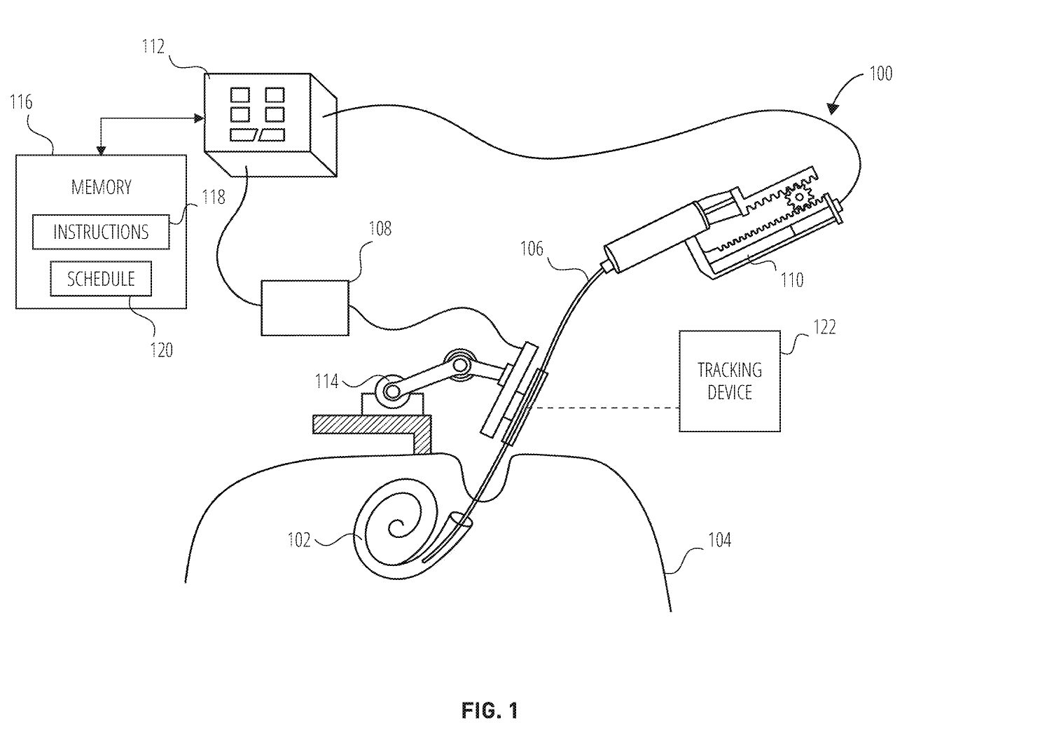

FIG. 1 illustrates an example of a system 100. The system 100 can be configured for synchronized instrument and fluid deployment. In examples, the system 100 can be used to perform medical procedures within cochlea 102 of a patient 104. For example, the system 100 can be operable by a clinician to perform medical procedures within an inner ear of the patient 104. The cochlea 102 can include a cochlea of the inner ear of the patient 104. The system 100 can include a medical instrument 106, an insertion actuator 108, an instrument actuator 110, a processor 112, an actuator positioner 114, a memory 116, instructions 118, a schedule 120, and a tracking device 122.

The medical instrument 106 can be configured to transfer fluid or biological matter relative to the cochlea 102 of the patient 104. The medical instrument 106 can include a needle, a sampling needle, a flexible needle, a flexible lumen, or the like. The medical instrument 106 can include a needle coupled to a lumen. The medical instrument 106 can be a medical device used by a clinician to perform a medical procedure within an ear of the patient 104.

The insertion actuator 108 can be operably coupled to the medical instrument 106. The insertion actuator 108 can be operable to translate the medical instrument 106 relative to the patient 104. For example, the insertion actuator 108 can be configured to insert the medical instrument 106 within the cochlea 102 (e.g., the cochlea, or other anatomy of the ear) of the patient 104. The insertion actuator 108 can be configured to retract the medical instrument 106 from the cochlea 102 of the patient 104. The insertion actuator 108 can be releasably coupled to the medical instrument 106.

The instrument actuator 110 can be operably coupled to the medical instrument 106. The instrument actuator 110 can be selectively configured to deploy a fluid (e.g., a viral vector, medication, stem cells, gene therapies, combinations thereof, or the like) stored at least partially within the medical instrument 106 into the patient 104 or withdraw biological matter (e.g., tissue, bacteria, perilymph fluid, combinations thereof, or the like) from the patient 104 into the medical instrument 106.

The actuator positioner 114 can be operably coupled to the insertion actuator 108. The actuator positioner 114 can be configured to adjust a position (e.g., an orientation, an angle, a distance to the patient 104, or the like) of the insertion actuator 108 to aid in the insertion of the medical instrument 106 into the cochlea 102 of the patient 104. The actuator positioner 114 can provide at least two degrees of freedom for adjusting the position of the insertion actuator 108 relative to the cochlea 102 of the patient 104. The actuator positioner 114 can be further configured to be coupled to the patient 104. For example, the actuator positioner 114 can be configured to be coupled to a skull or a cheek of the patient 104.

The tracking device 122 can be operably coupled to the medical instrument 106 via the actuator positioner 114. In examples, the tracking device 122 can be coupled directly to the medical instrument 106. In examples, the tracking device 122 can be operably coupled to the instrument actuator 110. The tracking device 122 can be configured to monitor an insertion depth of the medical instrument 106 relative to the cochlea 102 of the patient 104. The tracking device 122 can include an encoder, a step counter, a linear variable differential transformer (LVDT), or combinations thereof. The tracking device 122 can be communicatively coupled to the processor 112 to provide position feedback data corresponding to a location of the medical instrument 106 relative to the cochlea 102. In examples, the step counter can include a program or an algorithm configured to count signals sent to one or more motors of the system to determine the insertion of the medical instrument within the patient. For example, each signal, or pulse, sent to a motor can be counted from the step counter.

The processor 112 can be configured to control the components of the system 100. The processor 112 can include processing circuitry. The processor 112 can be communicatively coupled to the insertion actuator 108, the instrument actuator 110, and the tracking device 122. The processor 112 can be coupled to the memory 116. The memory 116 can store the schedule 120 and the instructions 118. The instructions 118, when executed by the processing circuitry, can configure the processing circuitry to synchronize the actuation of the insertion actuator 108 and the instrument actuator 110 according to the schedule 120. The schedule 120 can coordinate translation of the medical instrument 106 with at least one of distribution of the fluid or withdrawal of the biological matter.

The instructions 118, when executed by the processing circuitry, can configure the processing circuitry to deploy, by transmitting a first actuation control signal to the insertion actuator 108, the medical instrument 106 a set distance within the cochlea 102 of the patient 104. The processor 112 can receive position feedback data from the tracking device 122 to determine when the medical instrument 106 has traversed the set distance. The instructions 118 can configure the processing circuitry to distribute, by transmitting a first fluid control signal to the instrument actuator 110, a first portion of the fluid (e.g., a viral vector, medication, stem cells, gene therapies, combinations thereof, or the like) into the cochlea 102 of the patient 104. The instructions 118 can configure the processing circuitry to translate, by transmitting a second actuation control signal to the insertion actuator 108, the medical instrument 106 a second set distance relative to the cochlea 102 of the patient 104. The instructions 118 can configure the processing circuitry to distribute, by transmitting a second fluid control signal to the instrument actuator 110, a second portion of the fluid into the cochlea 102 of the patient 104.

The instructions 118 can configure the processing circuitry to deploy, by transmitting a third actuation control signal to the insertion actuator 108, the medical instrument 106 a third set distance within the cochlea 102 of the patient 104. The instructions 118 can configure the processing circuitry to withdraw, by transmitting a first suction control signal to the instrument actuator 110, biological matter from within the cochlea 102 of the patient 104. The instructions 118 can configure the processing circuitry to retract, by transmitting a fourth actuation control signal to the insertion actuator 108, the medical instrument 106 from the cochlea 102 of the patient 104.

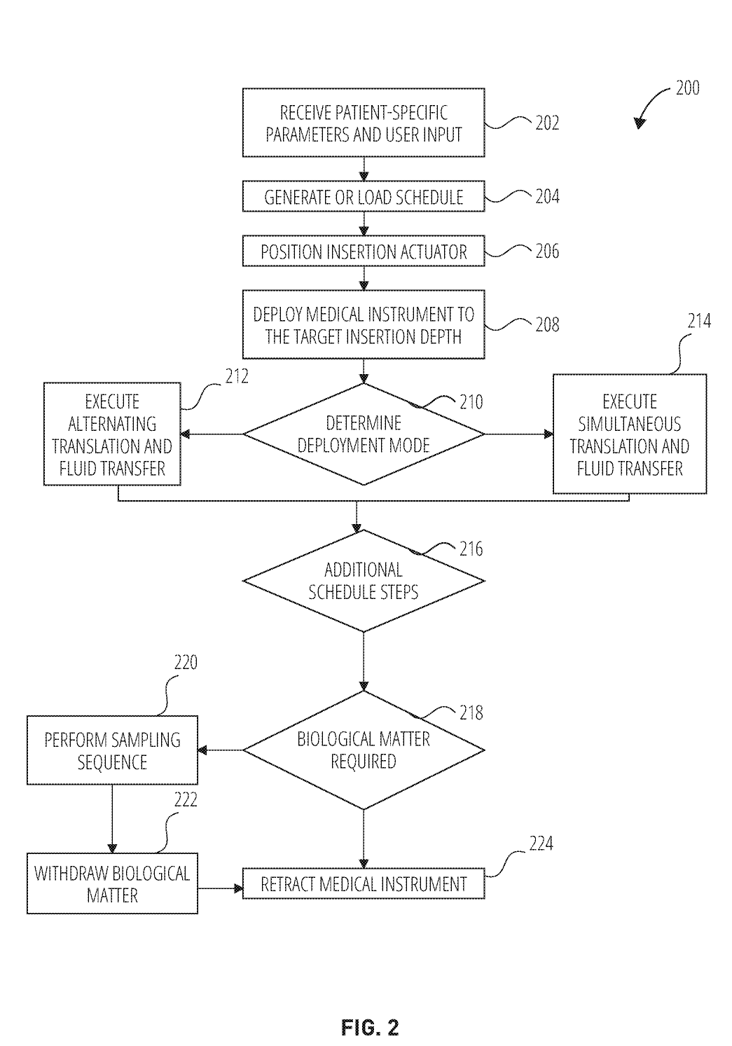

FIG. 2 illustrates a flowchart of an example method 200 for synchronized instrument and fluid deployment. The method 200 can be performed using the system 100 described with reference to FIG. 1. The method 200 can be performed by the processor 112 executing the instructions 118 stored in the memory 116.

At operation 202, the system 100 can receive patient-specific parameters and user input. The patient-specific parameters can include anatomical measurements, patient imaging data, disease process information, diagnostic data, genetic information, or combinations thereof. The user input can include clinician-defined parameters for the medical procedure, such as a target insertion depth, a fluid volume, a fluid transfer rate, or the like. The patient-specific parameters and user input can be received via a user interface communicatively coupled to the processor 112.

At operation 204, the system 100 can generate or load a schedule 120. The schedule 120 can be generated by the processor 112 based on the patient-specific parameters and user input received at operation 202. The schedule 120 can be loaded from a predetermined data file stored in the memory 116 or received from an external source via a network interface. The schedule 120 can specify fluid volumes to be distributed over defined time intervals, translation distances to be traversed over defined time intervals, cycle counts, mode indicators specifying simultaneous or alternating translation and fluid transfer, or combinations thereof.

At operation 206, the system 100 can position the insertion actuator 108. The actuator positioner 114 can adjust the position of the insertion actuator 108 relative to the cochlea 102 of the patient 104. The actuator positioner 114 can provide at least two degrees of freedom for adjusting the orientation, angle, or distance of the insertion actuator 108 relative to the cochlea 102. The actuator positioner 114 can be coupled to the patient 104, such as to a skull or a cheek of the patient 104, to stabilize the insertion actuator 108 during the medical procedure.

At operation 208, the system 100 can deploy the medical instrument 106 to a target insertion depth within the cochlea 102 of the patient 104. The processor 112 can transmit a first actuation control signal to the insertion actuator 108 to insert the medical instrument 106 a set distance within the cochlea 102. The tracking device 122 can monitor the insertion depth of the medical instrument 106 relative to the cochlea 102 during deployment. The processor 112 can receive position feedback data from the tracking device 122 to determine when the medical instrument 106 has reached the target insertion depth specified by the schedule 120. The processor 112 can adjust or terminate the first actuation control signal based on the position feedback data from the tracking device 122.

At operation 210, the system 100 can determine a deployment mode. The deployment mode can be specified by the schedule 120. The deployment mode can indicate whether the system 100 is to execute alternating translation and fluid transfer or simultaneous translation and fluid transfer. The processor 112 can read a mode indicator from the schedule 120 to determine the deployment mode for a given step or cycle of the schedule 120.

At operation 212, the system 100 can execute alternating translation and fluid transfer. In the alternating mode, the processor 112 can transmit a fluid control signal to the instrument actuator 110 to distribute a portion of the fluid into the cochlea 102 of the patient 104 while the medical instrument 106 is held at a given position. After distribution of the portion of fluid, the processor 112 can transmit an actuation control signal to the insertion actuator 108 to translate the medical instrument 106 a set distance relative to the cochlea 102. The tracking device 122 can monitor the position of the medical instrument 106 during translation and provide position feedback data to the processor 112. The processor 112 can confirm that the medical instrument 106 has traversed the set distance based on the position feedback data from the tracking device 122 before transmitting a subsequent fluid control signal to distribute a next portion of the fluid. This alternating sequence of fluid distribution and translation can be repeated according to the schedule 120.

At operation 214, the system 100 can execute simultaneous translation and fluid transfer. In the simultaneous mode, the processor 112 can concurrently transmit an actuation control signal to the insertion actuator 108 and a fluid control signal to the instrument actuator 110. The insertion actuator 108 can translate the medical instrument 106 relative to the cochlea 102 while the instrument actuator 110 distributes fluid into the cochlea 102 at the same time. The tracking device 122 can monitor the position of the medical instrument 106 during the simultaneous translation and fluid transfer. The processor 112 can receive position feedback data from the tracking device 122 to regulate the rate of translation and the rate of fluid distribution according to the schedule 120. The simultaneous translation and fluid distribution can continue until the schedule 120 indicates that the fluid volume or translation distance for the current cycle has been reached.

At operation 216, the system 100 can determine whether additional schedule steps remain. The processor 112 can evaluate the schedule 120 to determine whether additional cycles of translation and fluid transfer are specified. If additional schedule steps remain, the method 200 can return to operation 210 to determine the deployment mode for the next cycle. If no additional schedule steps remain, the method 200 can proceed to operation 218.

At operation 218, the system 100 can determine whether biological matter is required. The schedule 120 or user input can indicate whether sampling of biological matter from the cochlea 102 is to be performed. If biological matter sampling is required, the method 200 can proceed to operation 220. If biological matter sampling is not required, the method 200 can proceed to operation 224.

At operation 220, the system 100 can perform a sampling sequence. The processor 112 can transmit an actuation control signal to the insertion actuator 108 to deploy the medical instrument 106 to a sampling depth within the cochlea 102 of the patient 104. The tracking device 122 can monitor the insertion depth of the medical instrument 106 during the sampling deployment and provide position feedback data to the processor 112.

At operation 222, the system 100 can withdraw biological matter from the cochlea 102 of the patient 104. The processor 112 can transmit a suction control signal to the instrument actuator 110 to withdraw biological matter (e.g., perilymph fluid, tissue, infection, or the like) from the cochlea 102 into the medical instrument 106. The withdrawal of biological matter can be performed according to a withdrawal schedule specified by the schedule 120. The withdrawal schedule can specify withdrawal amounts over defined time periods, translation distances during withdrawal, simultaneous translation and withdrawal, or alternating patterns of translation and withdrawal.

At operation 224, the system 100 can retract the medical instrument 106 from the cochlea 102 of the patient 104. The processor 112 can transmit an actuation control signal to the insertion actuator 108 to retract the medical instrument 106. The tracking device 122 can monitor the retraction of the medical instrument 106 and provide position feedback data to the processor 112 to confirm that the medical instrument 106 has been fully retracted from the cochlea 102.

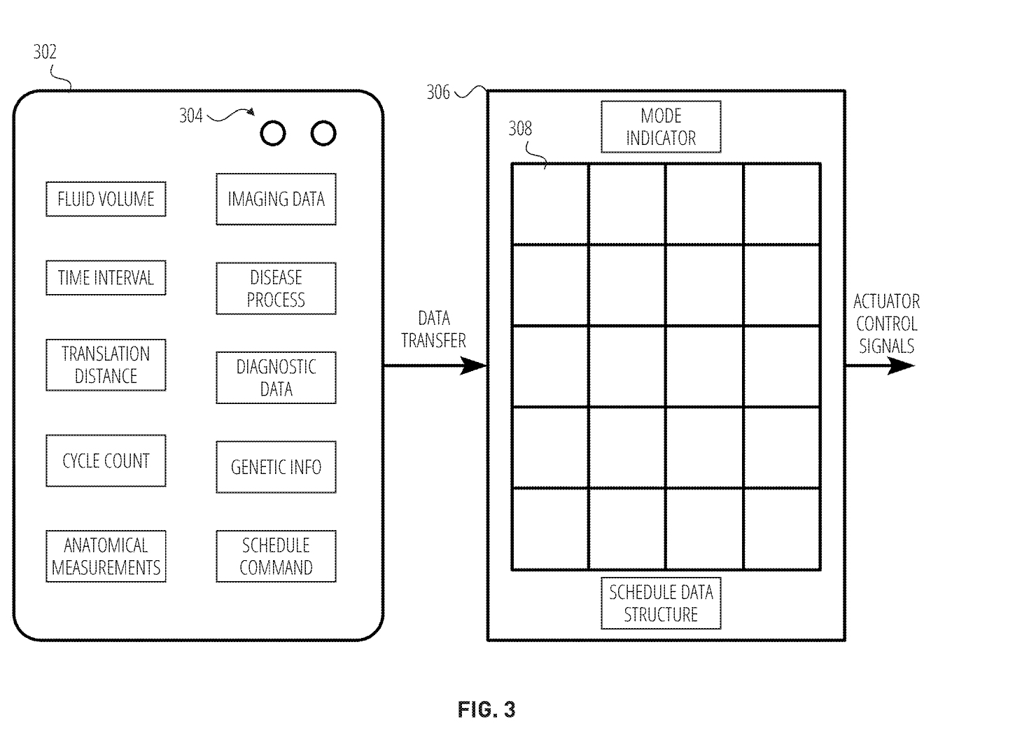

FIG. 3 illustrates a block diagram of an example of a user interface and a schedule data structure for synchronized instrument and fluid deployment. FIG. 3 includes a user interface 302, mode selection toggles 304, a schedule data structure 306, and a schedule table 308.

The user interface 302 can be communicatively coupled to the processor 112 (FIG. 1). The user interface 302 can enable a user to define the schedule 120 (FIG. 1), load the schedule 120 from a data file, or generate the schedule 120 based on parameters entered by the user. The user interface 302 can be displayed on a display unit, such as a touch-screen display, a monitor, or another output device. The user interface 302 can receive input from the user via an input device such as a keyboard, a mouse, a touch screen, or the like.

The user interface 302 can include a set of input fields for receiving schedule parameters from the user. The schedule parameters can define the operational characteristics of each step of the schedule 120. The schedule parameters can include a fluid volume field, a time interval field, a translation distance field, a cycle count field, and an anatomical measurements field. The fluid volume field can receive a value specifying an amount of fluid to be transferred during a step of the schedule 120. The fluid volume field can specify a volume of fluid to be distributed into the anatomy of the patient 104 (FIG. 1), such as a volume of a viral vector, medication, stem cells, gene therapies, or the like. The fluid volume field can also specify a volume of biological matter to be withdrawn from the anatomy of the patient 104, such as a volume of perilymph fluid, tissue, infection, or the like. The direction of fluid transfer for a given step can be indicated by the schedule command input or the mode selection toggles 304. The amount of fluid can be specified in units of volume such as microliters or nanoliters. The time interval field can receive a value specifying a duration over which a step of the schedule 120 is to be performed. The duration can be specified in units of time such as seconds or minutes. The translation distance field can receive a value specifying a distance over which the medical instrument 106 (FIG. 1) is to be translated during a step of the schedule 120. The distance can be specified in units of length such as millimeters or micrometers. The cycle count field can receive a value specifying a number of times a step or a sequence of steps of the schedule 120 is to be repeated. The anatomical measurements field can receive values specifying dimensions or distances within the anatomy of the patient 104 (FIG. 1), such as a length of the cochlea 102 (FIG. 1), a diameter of the cochlea 102, a depth of insertion available within the cochlea 102, or the like. The anatomical measurements can be entered manually by the user or can be populated from patient imaging data or diagnostic data.

The user interface 302 can also include input fields for receiving patient-specific parameters from the user. The patient-specific parameters can be used by the processor 112 to generate the schedule 120 or to adjust the schedule parameters based on characteristics of the patient 104. The patient-specific parameter input fields can include an imaging data field, a disease process field, a diagnostic data field, and a genetic information field. The imaging data field can receive or reference patient imaging data such as computed tomography (CT) scans, magnetic resonance imaging (MRI) data, or other imaging modalities that depict the anatomy of the patient 104. The disease process field can receive information describing a disease process affecting the patient 104, such as a type of hearing loss, a location of a lesion within the cochlea 102, a stage of disease progression, or the like. The diagnostic data field can receive diagnostic information about the patient 104, such as audiometric test results, vestibular function test results, or other clinical measurements. The genetic information field can receive genetic data about the patient 104, such as gene mutation information, genetic predisposition data, or information about a target gene for gene therapy delivery. The processor 112 can use the patient-specific parameters to calculate schedule parameters, select a schedule template, or adjust a loaded schedule to account for characteristics of the patient 104.

The user interface 302 can also include a schedule command input. The schedule command input can enable the user to instruct the processor 112 to perform an action related to the schedule 120. For example, the schedule command input can include a generate command that instructs the processor 112 to generate the schedule 120 based on the schedule parameters and patient-specific parameters entered via the user interface 302. The schedule command input can include a load command that instructs the processor 112 to load the schedule 120 from a predetermined data file stored in the memory 116 (FIG. 1) or received from an external source. The schedule command input can include an execute command that instructs the processor 112 to initiate execution of the schedule 120. The schedule command input can include a save command that instructs the processor 112 to store the schedule 120 to the memory 116 or to an external storage device. The schedule command input can be implemented as a button, a dropdown menu, a text input, or the like on the user interface 302.

The mode selection toggles 304 can be displayed on the user interface 302. The mode selection toggles 304 can enable the user to select a deployment mode for the schedule 120. The deployment mode can specify whether translation of the medical instrument 106 and distribution of the fluid or withdrawal of biological matter are to be performed simultaneously or in an alternating pattern. For example, a first toggle of the mode selection toggles 304 can correspond to an alternating mode in which the processor 112 sequences translation and fluid transfer operations in an alternating pattern. A second toggle of the mode selection toggles 304 can correspond to a simultaneous mode in which the processor 112 performs translation and fluid transfer operations concurrently. A third toggle of the mode selection toggles 304 can correspond to a withdrawal mode in which the processor 112 sequences or concurrently performs translation and biological matter withdrawal operations. The user can select one or more of the mode selection toggles 304 to set the deployment mode and the direction of fluid transfer for one or more steps of the schedule 120. In examples, the mode selection toggles 304 can be implemented as radio buttons, toggle switches, checkboxes, or the like. In examples, the mode selection toggles 304 can enable the user to set a single deployment mode that applies to all steps of the schedule 120. In other examples, the mode selection toggles 304 can enable the user to set a deployment mode for individual steps of the schedule 120.

The schedule data structure 306 can be stored in the memory 116. The schedule data structure 306 can represent a data format in which the schedule 120 is organized and stored. The schedule parameters, patient-specific parameters, and mode selections entered via the user interface 302 can be transferred to the schedule data structure 306 via a data transfer path. The data transfer path can communicatively couple the user interface 302 to the schedule data structure 306. The data transfer path can represent a communication link between the user interface 302 and the memory 116 in which the schedule data structure 306 is stored. The data transfer path can be a wired connection, a wireless connection, a bus, a software interface, or the like.

The schedule data structure 306 can include the schedule table 308. The schedule table 308 can include a plurality of rows and a plurality of columns. Each row of the schedule table 308 can correspond to a step of the schedule 120. Each column of the schedule table 308 can correspond to a parameter of the schedule 120. The columns of the schedule table 308 can include a fluid volume column specifying the amount of fluid to be distributed or withdrawn during each step, a time interval column specifying the duration of each step, a translation distance column specifying the distance the medical instrument 106 is to be translated during each step, and a cycle count column specifying the number of repetitions for each step. The schedule table 308 can also include a mode indicator column. The mode indicator column can include a mode indicator for each row. The mode indicator can specify whether the corresponding step is to be performed in the alternating mode or the simultaneous mode, as selected via the mode selection toggles 304 on the user interface 302. The mode indicator can also specify the direction of fluid transfer for the corresponding step, such as distribution of fluid into the anatomy or withdrawal of biological matter from the anatomy. The schedule data structure 306 can also store patient-specific parameters such as anatomical measurements, imaging data, disease process information, diagnostic data, and genetic information that were used to generate or populate the schedule table 308. The schedule data structure 306 can be stored as a data file, a database record, a data array, a linked list, or another data format in the memory 116.

The processor 112 can read the schedule data structure 306 from the memory 116 and can generate actuator control signals based on the parameters and mode indicators stored in the schedule table 308. The actuator control signals can be output from the processor 112 via the schedule data structure 306. The actuator control signals can be transmitted from the processor 112 to the insertion actuator 108 (FIG. 1) and the instrument actuator 110 (FIG. 1) to synchronize translation of the medical instrument 106 with the distribution of the fluid or withdrawal of biological matter according to the schedule 120. For each row of the schedule table 308, the processor 112 can read the mode indicator to determine whether to transmit the actuation control signals and the fluid control signals in an alternating pattern or concurrently. The processor 112 can also read the mode indicator to determine whether to transmit a fluid control signal for distribution or a suction control signal for withdrawal for the corresponding step. The processor 112 can read the fluid volume, time interval, and translation distance values from each row to determine the magnitude and timing of the actuation control signals and the fluid control signals. The processor 112 can read the cycle count value from each row to determine whether to repeat the step before proceeding to the next row of the schedule table 308.

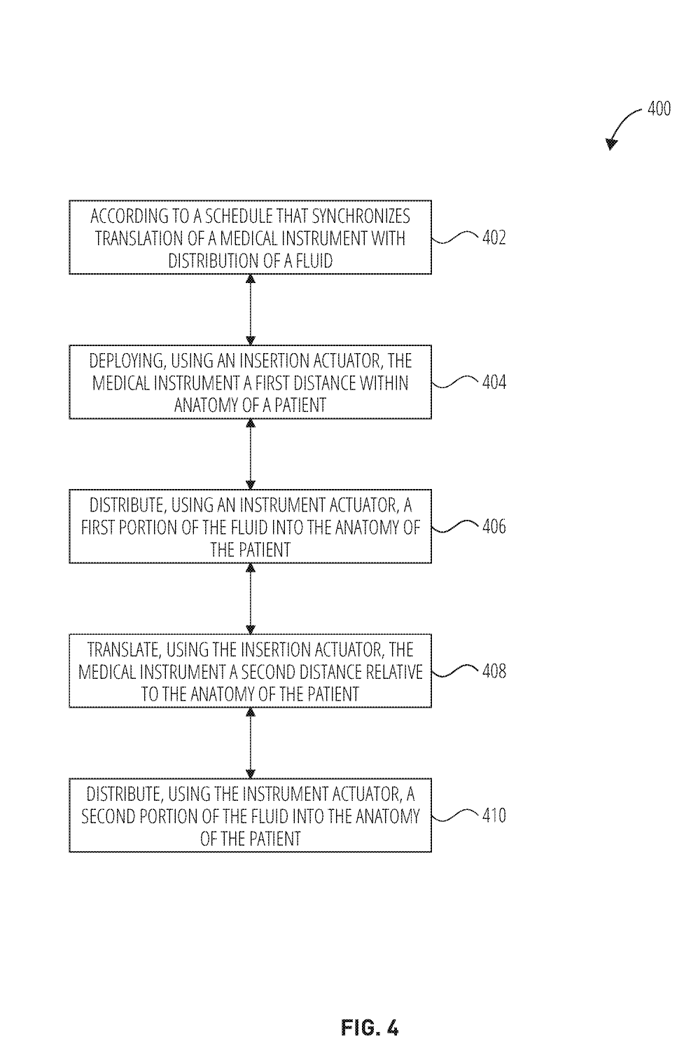

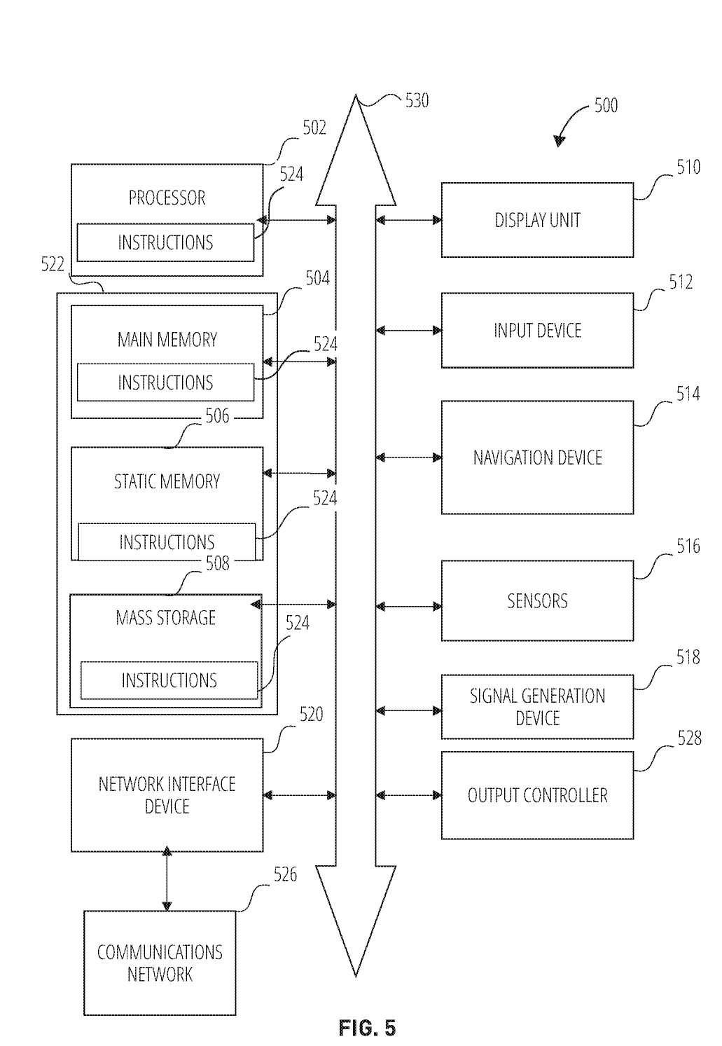

FIG. 4 illustrates a flowchart of an example method 400 for synchronized instrument and fluid deployment. Although the example method 400 depicts a particular sequence of operations, the sequence can be altered without departing from the scope of the present disclosure. For example, some of the operations depicted can be performed in parallel or in a different sequence that does not materially affect the function of the method 400. In other examples, different components of an example device or system that implements the method 400 can perform functions at substantially the same time or in a specific sequence. The method 400 can be performed by the system 100 (FIG. 1), by the machine 500 (FIG. 5), or by another computing device or system configured to control an insertion actuator and an instrument actuator.

At operation 402, the method 400 can include operating according to a schedule that synchronizes translation of a medical instrument with distribution of a fluid. The schedule can be the schedule 120 (FIG. 1) stored in the memory 116 (FIG. 1). The schedule can be generated, loaded, or defined via the user interface 302 (FIG. 3) as described with respect to FIG. 3. The schedule can coordinate translation of the medical instrument 106 (FIG. 1) with at least one of distribution of the fluid or withdrawal of biological matter. The schedule can specify, for each step, at least one of a fluid volume to be distributed, a time interval over which the step is to be performed, a translation distance over which the medical instrument 106 is to be translated, a cycle count specifying a number of repetitions, or a mode indicator specifying whether translation and fluid transfer are to be performed simultaneously or in an alternating pattern. Operations 404 through 410 can be performed according to the schedule.

At operation 404, the method 400 can include deploying, using an insertion actuator, the medical instrument a first distance within anatomy of a patient. The processor 112 (FIG. 1) can transmit a first actuation control signal to the insertion actuator 108 (FIG. 1) to translate the medical instrument 106 the first distance within the anatomy of the patient 104 (FIG. 1). The first distance can correspond to a target insertion depth specified by the schedule. For example, the first distance can correspond to a depth within the cochlea 102 (FIG. 1) of the inner ear of the patient 104. The insertion actuator 108 can translate the medical instrument 106 in a direction toward the anatomy of the patient 104 in response to the first actuation control signal. A tracking device operably coupled to the medical instrument 106 or the insertion actuator 108 can monitor the insertion depth of the medical instrument 106 relative to the patient 104 during the deploying operation. The tracking device can include at least one of an encoder, a step counter, or a linear variable differential transformer (LVDT). The tracking device can provide feedback to the processor 112 to confirm that the medical instrument 106 has reached the first distance. The medical instrument 106 can include at least one of a flexible needle, a needle coupled to a lumen, or a flexible lumen. The insertion actuator 108 can be releasably coupled to the medical instrument 106.

At operation 406, the method 400 can include distributing, using an instrument actuator, a first portion of the fluid into the anatomy of the patient. The processor 112 can transmit a first fluid control signal to the instrument actuator 110 (FIG. 1) to distribute the first portion of the fluid into the anatomy of the patient 104. The first portion of the fluid can be a volume specified by the schedule for the current step. The fluid can include a viral vector, a gene therapy agent, stem cells, a medication, combinations thereof, or the like. The instrument actuator 110 can drive a syringe, a pump, or another fluid delivery mechanism to deploy the first portion of the fluid through the medical instrument 106 and into the anatomy of the patient 104. The rate of distribution can be specified by the schedule. For example, the schedule can specify a fluid volume to be distributed over a defined time interval. The rate of distribution can be controlled to limit turbulence within the anatomy of the patient 104, such as within perilymph fluid in the cochlea 102.

At operation 408, the method 400 can include translating, using the insertion actuator, the medical instrument a second distance relative to the anatomy of the patient. The processor 112 can transmit a second actuation control signal to the insertion actuator 108 to translate the medical instrument 106 the second distance relative to the anatomy of the patient 104. The second distance can correspond to a retraction distance specified by the schedule. For example, the insertion actuator 108 can retract the medical instrument 106 the second distance in a direction away from the anatomy of the patient 104, such that the distal end of the medical instrument 106 is repositioned at a different location within the cochlea 102. The second distance can be a controlled, incremental distance specified by the schedule to enable distribution of the fluid at multiple locations within the anatomy of the patient 104. The tracking device operably coupled to the medical instrument 106 or the insertion actuator 108 can monitor the position of the medical instrument 106 during the translating operation and can provide feedback to the processor 112 to confirm that the medical instrument 106 has been translated the second distance. In examples, the translating at operation 408 can include inserting the medical instrument 106 a further distance into the anatomy of the patient 104 rather than retracting, depending on the schedule.

At operation 410, the method 400 can include distributing, using the instrument actuator, a second portion of the fluid into the anatomy of the patient. The processor 112 can transmit a second fluid control signal to the instrument actuator 110 to distribute the second portion of the fluid into the anatomy of the patient 104. The second portion of the fluid can be a volume specified by the schedule for the current step. The second portion can be the same volume as the first portion or a different volume, as specified by the schedule. Because the medical instrument 106 has been translated the second distance at operation 408, the second portion of the fluid can be distributed at a different location within the anatomy of the patient 104 than the first portion. The rate of distribution of the second portion can be specified by the schedule and can be controlled to limit turbulence within the anatomy of the patient 104.

In examples, the method 400 can repeat operations 408 and 410 for additional cycles as specified by the schedule, as described with respect to operation 216 of the method 200 (FIG. 2). Each cycle can translate the medical instrument 106 an additional distance and distribute an additional portion of the fluid at a different location within the anatomy of the patient 104. The number of cycles, the translation distance for each cycle, and the fluid volume for each cycle can each be specified by the schedule. By distributing portions of the fluid at multiple locations along a path within the anatomy of the patient 104, the method 400 can distribute the fluid throughout the anatomy rather than at a single location.

In examples, the method 400 can further include deploying the medical instrument 106 a third distance within the anatomy of the patient 104, withdrawing biological matter from the anatomy of the patient 104, and retracting the medical instrument 106 from the anatomy of the patient 104, as described with respect to operations 220, 222, and 224 of the method 200 (FIG. 2). The biological matter can include perilymph fluid, tissue, bacteria, combinations thereof, or the like. The deploying, withdrawing, and retracting can be performed according to the schedule.

FIG. 5 illustrates a block diagram of an example machine 500 upon which any one or more of the techniques (e.g., methodologies) discussed herein can be performed. Examples, as described herein, can include, or can operate by, logic or a number of components, or mechanisms in the machine 500. Circuitry (e.g., processing circuitry) is a collection of circuits implemented in tangible entities of the machine 500 that include hardware (e.g., simple circuits, gates, logic, etc.). Circuitry membership can be flexible over time. Circuitries include members that can, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.), including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional examples of these components with respect to the machine 500 follow.

In alternative examples, the machine 500 can operate as a standalone device or can be connected (e.g., networked) to other machines. In a networked deployment, the machine 500 can operate in the capacity of a server machine, a client machine, or both in server-client network environments. In an example, the machine 500 can act as a peer machine in peer-to-peer (P2P) (or other distributed) network environment. The machine 500 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 500 can include a hardware processor 502 (e.g., a central processing unit (CPU), a graphics processing unit (GPU), a hardware processor core, or any combination thereof), a main memory 504, a static memory 506 (e.g., memory or storage for firmware, microcode, a basic-input-output (BIOS)), and mass storage 508 (e.g., hard drives, tape drives, flash storage, or other block devices) some or all of which can communicate with each other via an interlink 530 (e.g., bus). The machine 500 can further include a display unit 510, an alphanumeric input device 512 (e.g., a keyboard), and a user interface (UI) navigation device 514 (e.g., a mouse). In examples, the display unit 510, input device 512, and UI navigation device 514 can be a touch screen display. The machine 500 can additionally include a signal generation device 518 (e.g., a speaker), a network interface device 520, and one or more sensors 516, such as a global positioning system (GPS) sensor, compass, accelerometer, or other sensor. The machine 500 can include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 502, the main memory 504, the static memory 506, or the mass storage 508 can be, or include, a machine-readable medium 522 on which is stored one or more sets of data structures or instructions 524 (e.g., software) embodying or utilized by any one or more of the techniques or functions described herein. The instructions 524 can also reside, completely or at least partially, within any of the registers of the processor 502, the main memory 504, the static memory 506, or the mass storage 508 during execution thereof by the machine 500. In an example, one or any combination of the hardware processor 502, the main memory 504, the static memory 506, or the mass storage 508 can constitute the machine-readable media 522. While the machine-readable medium 522 is illustrated as a single medium, the term “machine-readable medium” can include a single medium or multiple media (e.g., a centralized or distributed database, and/or associated caches and servers) configured to store the one or more instructions 524.

The term “machine-readable medium” can include any medium that is capable of storing, encoding, or carrying instructions for execution by the machine 500 and that cause the machine 500 to perform any one or more of the techniques of the present disclosure, or that is capable of storing, encoding, or carrying data structures used by or associated with such instructions. Non-limiting machine-readable medium examples can include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine-readable medium 522 can be representative of the instructions 524, such as the instructions 524 themselves or a format from which the instructions 524 can be derived. This format from which the instructions 524 can be derived can include source code, encoded instructions (e.g., in compressed or encrypted form), packaged instructions (e.g., split into multiple packages), or the like. The information representative of the instructions 524 in the machine-readable medium 522 can be processed by processing circuitry into the instructions to implement any of the operations discussed herein. For example, deriving the instructions 524 from the information (e.g., processing by the processing circuitry) can include: compiling (e.g., from source code, object code, etc.), interpreting, loading, organizing (e.g., dynamically or statically linking), encoding, decoding, encrypting, decrypting, packaging, unpackaging, or otherwise manipulating the information into the instructions 524.

In an example, the derivation of the instructions 524 can include assembly, compilation, or interpretation of the information (e.g., by the processing circuitry) to create the instructions 524 from some intermediate or preprocessed format provided by the machine-readable medium 522. The information, when provided in multiple parts, can be combined, unpacked, and modified to create the instructions 524. For example, the information can be in multiple compressed source code packages (or object code, or binary executable code, etc.) on one or several remote servers. The source code packages can be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 524 can be further transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others). In an example, the network interface device 520 can include one or more physical jacks (e.g., Ethernet, coaxial, or phone jacks) or one or more antennas to connect to the communications network 526. In an example, the network interface device 520 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 500, and includes digital or analog communications signals or other intangible medium to facilitate communication of such software. A transmission medium is a machine-readable medium.

The following, non-limiting examples, detail certain aspects of the present subject matter to solve the challenges and provide the benefits discussed herein, among others.

Example 1 is a system for synchronized instrument and fluid deployment, the system comprising: a medical instrument for use within anatomy of a patient; an insertion actuator operably coupled to the medical instrument, the insertion actuator operable to translate the medical instrument relative to the patient; an instrument actuator operably coupled to the medical instrument, the instrument actuator selectively configured to deploy a fluid stored at least partially within the medical instrument into the patient or withdraw biological matter from the patient into the medical instrument; processing circuitry; and a memory storing a schedule and instructions that, when executed by the processing circuitry, configure the processing circuitry to synchronize actuation of the insertion actuator and the instrument actuator according to the schedule, the schedule coordinating translation of the medical instrument with at least one of distribution of the fluid or withdrawal of the biological matter.

In Example 2, the subject matter of Example 1 optionally includes a tracking device operably coupled to the medical instrument or the insertion actuator, the tracking device configured to monitor an insertion depth of the medical instrument relative to the patient, the tracking device including at least one of an encoder, a step counter, or a linear variable differential transformer.

In Example 3, the subject matter of any one or more of Examples 1–2 optionally include wherein the schedule specifies at least one of simultaneous translation of the medical instrument and distribution of the fluid, simultaneous translation of the medical instrument and withdrawal of the biological matter, alternating patterns of translation of the medical instrument and distribution of the fluid, or alternating patterns of translation of the medical instrument and withdrawal of the biological matter.

In Example 4, the subject matter of any one or more of Examples 1–3 optionally include wherein the insertion actuator is releasably coupled to the medical instrument.

In Example 5, the subject matter of any one or more of Examples 1–4 optionally include wherein the medical instrument includes at least one of a flexible needle, a needle coupled to a lumen, or a flexible lumen.

In Example 6, the subject matter of any one or more of Examples 1–5 optionally include wherein the anatomy includes a cochlea of an inner ear of the patient.

In Example 7, the subject matter of any one or more of Examples 1–6 optionally include wherein the fluid includes a viral vector.

In Example 8, the subject matter of any one or more of Examples 1–7 optionally include wherein the biological matter includes perilymph fluid.

In Example 9, the subject matter of any one or more of Examples 1–8 optionally include wherein the instructions further configure the processing circuitry to: deploy, by transmitting a third actuation control signal to the insertion actuator, the medical instrument a third set distance within anatomy of the patient; withdraw, by transmitting a first suction control signal to the instrument actuator, biological matter from within the anatomy of the patient; and retract, by transmitting a fourth actuation control signal to the insertion actuator, the medical instrument from the anatomy of the patient.

In Example 10, the subject matter of Example 9 optionally includes wherein the biological matter includes perilymph fluid.

In Example 11, the subject matter of any one or more of Examples 1–10 optionally include wherein the schedule specifies at least one of a fluid volume to be distributed over a defined time interval or a translation distance to be traversed over a defined time interval.

In Example 12, the subject matter of any one or more of Examples 1–11 optionally include an actuator positioner operably coupled to the insertion actuator, the actuator positioner providing at least two degrees of freedom for adjusting a position of the insertion actuator relative to the patient, the actuator positioner configured to be coupled to the patient.

In Example 13, the subject matter of any one or more of Examples 1–12 optionally include a user interface configured to at least one of define the schedule, load the schedule from a data file, or generate the schedule based on patient-specific parameters, the patient-specific parameters including at least one of anatomical measurements, patient imaging data, disease process information, diagnostic data, or genetic information.

Example 14 is a method for synchronized instrument and fluid deployment, the method comprising: according to a schedule that synchronizes translation of a medical instrument with distribution of a fluid: deploying, using an insertion actuator, the medical instrument a first distance within anatomy of a patient; distributing, using an instrument actuator, a first portion of the fluid into the anatomy of the patient; translating, using the insertion actuator, the medical instrument a second distance relative to the anatomy of the patient; and distributing, using the instrument actuator, a second portion of the fluid into the anatomy of the patient.

In Example 15, the subject matter of Example 14 optionally includes monitoring an insertion depth of the medical instrument relative to the anatomy of the patient using at least one of an encoder, a step counter, or a linear variable differential transformer.

In Example 16, the subject matter of any one or more of Examples 14–15 optionally include wherein the deploying and the distributing are performed at least one of simultaneously or in an alternating pattern.

In Example 17, the subject matter of any one or more of Examples 14–16 optionally include wherein the anatomy includes a cochlea of an inner ear of the patient, and wherein the fluid includes a viral vector.

In Example 18, the subject matter of any one or more of Examples 14–17 optionally include deploying, using the insertion actuator, the medical instrument a third distance within the anatomy of the patient; withdrawing, using the instrument actuator, biological matter from the anatomy of the patient; and retracting, using the insertion actuator, the medical instrument from the anatomy of the patient; wherein the deploying, withdrawing, and retracting are performed according to the schedule.

In Example 19, the subject matter of any one or more of Examples 14–18 optionally include receiving or generating the schedule based on patient-specific parameters including at least one of anatomical measurements, patient imaging data, disease process information, diagnostic data, or genetic information.

Example 20 is a non-transitory computer-readable medium storing instructions that, when executed by processing circuitry, cause the processing circuitry to: according to a schedule stored in a memory, the schedule coordinating translation of a medical instrument with at least one of distribution of a fluid or withdrawal of biological matter: transmit a first actuation control signal to an insertion actuator to deploy the medical instrument a first distance within anatomy of a patient; transmit a first fluid control signal to an instrument actuator to distribute a first portion of the fluid into the anatomy of the patient; transmit a second actuation control signal to the insertion actuator to translate the medical instrument a second distance relative to the anatomy of the patient; and transmit a second fluid control signal to the instrument actuator to distribute a second portion of the fluid into the anatomy of the patient.

In Example 21, the subject matter of Example 20 optionally includes wherein the schedule specifies at least one of simultaneous translation of the medical instrument and distribution of the fluid, simultaneous translation of the medical instrument and withdrawal of the biological matter, alternating patterns of translation of the medical instrument and distribution of the fluid, or alternating patterns of translation of the medical instrument and withdrawal of the biological matter.

In Example 22, the subject matter of any one or more of Examples 20–21 optionally include wherein the instructions further cause the processing circuitry to generate the schedule based on patient-specific parameters including at least one of anatomical measurements, patient imaging data, disease process information, diagnostic data, or genetic information.

Example 23 includes a method, device, system, or the like, including any element of any example of Examples 1–22.

The above detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A system for synchronized instrument and fluid deployment, the system comprising:
a medical instrument for use within anatomy of a patient;
an insertion actuator operably coupled to the medical instrument, the insertion actuator operable to translate the medical instrument relative to the patient;
an instrument actuator operably coupled to the medical instrument, the instrument actuator selectively configured to deploy a fluid stored at least partially within the medical instrument into the patient or withdraw biological matter from the patient into the medical instrument;
processing circuitry communicatively coupled to the insertion actuator and the instrument actuator; and
a memory storing a schedule and instructions that, when executed by the processing circuitry, configure the processing circuitry to synchronize actuation of the insertion actuator and the instrument actuator according to the schedule, the schedule coordinating translation of the medical instrument with at least one of distribution of the fluid or withdrawal of the biological matter.

2. The system of claim 1, further comprising a tracking device operably coupled to the medical instrument or the insertion actuator, the tracking device configured to monitor an insertion depth of the medical instrument relative to the patient, the tracking device including at least one of an encoder, a step counter, or a linear variable differential transformer.

3. The system of claim 1, wherein the schedule specifies at least one of simultaneous translation of the medical instrument and distribution of the fluid, simultaneous translation of the medical instrument and withdrawal of the biological matter, alternating patterns of translation of the medical instrument and distribution of the fluid, or alternating patterns of translation of the medical instrument and withdrawal of the biological matter.

4. The system of claim 1, wherein the insertion actuator is releasably coupled to the medical instrument.

5. The system of claim 1, wherein the medical instrument includes at least one of a flexible needle, a needle coupled to a lumen, or a flexible lumen.

6. The system of claim 1, wherein the anatomy includes a cochlea of an inner ear of the patient.

7. The system of claim 1, wherein the fluid includes a viral vector.

8. The system of claim 1, wherein the biological matter includes perilymph fluid.

9. The system of claim 1, wherein the instructions further configure the processing circuitry to:
deploy, by transmitting a third actuation control signal to the insertion actuator, the medical instrument a third set distance within anatomy of the patient;
withdraw, by transmitting a first suction control signal to the instrument actuator, biological matter from within the anatomy of the patient; and
retract, by transmitting a fourth actuation control signal to the insertion actuator, the medical instrument from the anatomy of the patient.

10. The system of claim 9, wherein the biological matter includes perilymph fluid.

11. The system of claim 1, wherein the schedule specifies at least one of a fluid volume to be distributed over a defined time interval or a translation distance to be traversed over a defined time interval.

12. The system of claim 1, further comprising an actuator positioner operably coupled to the insertion actuator, the actuator positioner providing at least two degrees of freedom for adjusting a position of the insertion actuator relative to the patient, the actuator positioner configured to be coupled to the patient.

13. The system of claim 1, further comprising a user interface configured to at least one of define the schedule, load the schedule from a data file, or generate the schedule based on patient-specific parameters, the patient-specific parameters including at least one of anatomical measurements, patient imaging data, disease process information, diagnostic data, or genetic information.

14. A method for synchronized instrument and fluid deployment, according to a schedule that synchronizes translation of a medical instrument with distribution of a fluid, the method comprising:
deploying, using an insertion actuator, the medical instrument a first distance within anatomy of a patient;
distributing, using an instrument actuator, a first portion of the fluid into the anatomy of the patient;
translating, using the insertion actuator, the medical instrument a second distance relative to the anatomy of the patient; and
distributing, using the instrument actuator, a second portion of the fluid into the anatomy of the patient.

15. The method of claim 14, further comprising monitoring an insertion depth of the medical instrument relative to the anatomy of the patient using at least one of an encoder, a step counter, or a linear variable differential transformer.

16. The method of claim 14, wherein the deploying and the distributing are performed at least one of simultaneously or in an alternating pattern.

17. The method of claim 14, wherein the anatomy includes a cochlea of an inner ear of the patient, and wherein the fluid includes a viral vector.

18. The method of claim 14, further comprising:
deploying, using the insertion actuator, the medical instrument a third distance within the anatomy of the patient;
withdrawing, using the instrument actuator, biological matter from the anatomy of the patient; and
retracting, using the insertion actuator, the medical instrument from the anatomy of the patient;
wherein the deploying, withdrawing, and retracting are performed according to the schedule.

19. The method of claim 14, further comprising receiving or generating the schedule based on patient-specific parameters including at least one of anatomical measurements, patient imaging data, disease process information, diagnostic data, or genetic information.

20. A non-transitory computer-readable medium storing instructions that, when executed by processing circuitry, cause the processing circuitry to:
access a first actuation control signal from a schedule stored in a memory, the schedule coordinating translation of a medical instrument with at least one of distribution of a fluid or withdrawal of biological matter;
transmit the first actuation control signal to an insertion actuator to deploy the medical instrument a first distance within anatomy of a patient;
access a first fluid control signal from the schedule stored in a memory;
transmit the first fluid control signal to an instrument actuator to distribute a first portion of the fluid into the anatomy of the patient;
access a second actuation control signal from the schedule;
transmit the second actuation control signal to the insertion actuator to translate the medical instrument a second distance relative to the anatomy of the patient;
access a second fluid control signal from the schedule; and
transmit the second fluid control signal to the instrument actuator to distribute a second portion of the fluid into the anatomy of the patient.

21. The non-transitory computer-readable medium of claim 20, wherein the schedule specifies at least one of simultaneous translation of the medical instrument and distribution of the fluid, simultaneous translation of the medical instrument and withdrawal of the biological matter, alternating patterns of translation of the medical instrument and distribution of the fluid, or alternating patterns of translation of the medical instrument and withdrawal of the biological matter.

22. The non-transitory computer-readable medium of claim 20, wherein the instructions further cause the processing circuitry to generate the schedule based on patient-specific parameters including at least one of anatomical measurements, patient imaging data, disease process information, diagnostic data, or genetic information.
